SEQUENCE LISTING (7 sequences)

SEQ ID NO: 1 - DNA, 4089 bp, Beta vulgaris
ctcgaggatccttgatttgtttttttgaccaatgagcattaagagtgatcaagcacatggatttcaaaatatcaatggttggatttttatcattctcaaatcaatggttaaagttaaatattctattttctaatttctaaatttataattctatactctagtattgatttgataagcaattagtaatctacttaaattcctaaactattattaataatataaattaataaaaattataaacttagaagtttaattattatttttaatttcaaacaagttgattgatgtaaatatgattatgaatgacgtgaaacagttaaggcaaagttgtagtttagtaactttaaaaattagtagtgttattataacttaatagaaattataaactttcaaaattaaattagtattattgatttttaagaaattattattctaaatttataaattcagaaatatgtgaacatgttacatgcggtgattacggattaatttaatgctaacatgtaagacgtaacacaataacagttaacctactactactacttgtataggtcttacattaatacttaagttattggttataaacttatattaatatacggttatttcttctattaagcgtagatttcttataaccttacttatacaattatttactattactcaactataatttgattgtattcagttaagtgaacttatataagttataacaagccttaatcagtactagaccttagcttatactatactacatatacgaattttcgcttttcatattagtatcttactgataagcaataaagtttactacgacatagtaacaataattaactacttgtttcaattcaattagatgatttgggttttgaaatgaatttaacttaaagatgattaccttagatagagtattgttatcttctattaagttaattacttaacttacattaatacatttgttgatattatccaatatccattatcttaaacgaacttacgccatcttgaattagctaatcgacttacattgatacatcgatctaatccaatattaacttgtatcaattcaaattagatgttttgagtttcaatatgcaacttaacttagagacgattaacttaaatgaagcattgttatattgaaccaatctactgagttatactattctacatctgtttgaattagatgctttcggttccgaaatgaattcaacttaaagatgattaacttaagtagagtactgttattatcttgaattaaacttagggtactgttaaactagatgttttgagtttcaatatgaatttaacttagagacgattaacttaaatggagtattgttatattgaaccaatctactgagttccattgttctacatttgttgcaactcaattagatgctttcagttccgaaatgaattcaacttaaaaagatgattaacttaagtagagtactattattatattgaactagtttatcgacttacattgatacactcattcaaataacaacatgtttcaatttaagtaaacgttttgagtttcaattactttatcgacttacgtcgatacattcatctaatctaatatcaattgtaatttcttttagtttggctaagatatgtgagtttcaaattgaatataaattgttagcgattaacttaaatggagtaatgttatcttgatttcacttatcgattcacatagataccttgagctaatccataatcaacttattttacgtcgattatagatatattaatccattgaactaatacactaagagtaagacactaacatagatttgtcttattccaaatcatatatattaagtaataaagtacactttaaccgatgtcactttcattcacgtttttatgaagtacaactatttttttgaggcaattgtggaagtgccgacaacacttatttgttgcattaatttttagccggacccgaaaacacctgaccaaaaccgacatgaagcacaatttttcccgaacaaatacaagagtgtgattaacttctacccgaaccgatgcgattattgtcgaagttatgtggttttttgtcaataactgaaataaaccgaaacgaaactgaaaataaccgcaacaaattttaactgatatggattcaaccgttggttggtttaactgtttggttaaaactgatttaaactgcaactgaactgaactgaaaatggaaagggaaaaaactgaattaaactgaactgaaaagtttgttaactgaactgaactgttaattatttcaactaatacaaactgaactgatatcaactgaactgtttggttataaccataactgaactgttgaactgaattgacacctctaattgcaacggcaacaatagaactgttacagtagcccaatctacaaaactaaattgacaagaactgttgcattagatactcctactacaacggtacgtaataatgtgttgcactagtaaattttagcctttgcctttgattaaaaatgttgtagtgacacatcacaataaaatcaaatctaatgaatatgctataaacaaacaattgttccaaaatcaactattcttacgaaatactcttttcgagggacgacatttttctgaaacaccctaagcgacgttattgactctaatacatagatgaaatatcaataaaaagggatttatatgaggtattctttaatttttttttaagaacatatgaggtaccatagtattttaaaaatcaatccatgtgcccctaacttcgtaatgtatccatcatatatatcccttttttttttaggggtccatcatatatatccttaatgcatgactatcaaacattagatatgtcttaatccccccctaacaatcaaccaaaattccaaaattggttactctatttgttcctattacaccttccaccaatctcaacaaattttggcattctggttaagtactcgaaggactacttaacgaaatatttgatgtttgttagccattaaggtttatgtgttggattcattataaatggtttgaaaagtttattatcatctttattaaaagttcaatagtacctcatgagaactctctataaatcaactatattagttatcaataataagatacattcggattctatttggcaacacatttaagtcacctcaaatgaataggtgttaaaagtttaagtcacctaaaataaatagtcatgattaaagtaagagcttaaatcaaatgtaagctaaaaaattgactgaaattaaattggtatcaagtataacatcttaatttaaggtgttaaaattttcaactaacttatcaattgagcttttttcatcaatttcacctctatttcagctttgtttttagcttatatttccaaaattgccaataggtctcatttatacctaagatattcaacaatcctggctctcttcctgctcgttcattccaaatttcccaaatgtaatcagattaaagcagtagtgatcggatcattgcagacttttcggtagaataactgaataagtgtactgagatagtgtcaaaatactgttatttaagttttctataagtactaaaatatgaattagttcaacatcatcttatattgtcggccaataagattgcccactgtataataatataaaacaaaatcacagaggtgatatcgccttaaaaaaaaaaaatcatagatgtgaattaatgtaaccgagcataaatcaactgtttcctttttttttaaaagagaaatcaactgtttcctagaaacaattgaatattatatggtggtttattgtttcaaatttcagaaaaacaaaaaaaaaaaagcctttgctacttacttacatagttgcatctatataaagggcacctagcccaaccattttacttacctaaactgtctgtgtttgcaccactctcatctaaaaaatctgtgaaagtgagcgtttcaaaattttcaaaagatgcacataacaggtaaattagaagttgaagttgatatcaattgtcatggtgatattttccatgaaatttttagcaccaggccacatgatgtctccaccatgtcacctgagaatatccatgg

SEQ ID NO: 2 - DNA, 4858 bp, Artificial sequence
  Arabidopsis thaliana and Beta vulgaris
ctcgaggatccttgatttgtttttttgaccaatgagcattaagagtgatcaagcacatggatttcaaaatatcaatggttggatttttatcattctcaaatcaatggttaaagttaaatattctattttctaatttctaaatttataattctatactctagtattgatttgataagcaattagtaatctacttaaattcctaaactattattaataatataaattaataaaaattataaacttagaagtttaattattatttttaatttcaaacaagttgattgatgtaaatatgattatgaatgacgtgaaacagttaaggcaaagttgtagtttagtaactttaaaaattagtagtgttattataacttaatagaaattataaactttcaaaattaaattagtattattgatttttaagaaattattattctaaatttataaattcagaaatatgtgaacatgttacatgcggtgattacggattaatttaatgctaacatgtaagacgtaacacaataacagttaacctactactactacttgtataggtcttacattaatacttaagttattggttataaacttatattaatatacggttatttcttctattaagcgtagatttcttataaccttacttatacaattatttactattactcaactataatttgattgtattcagttaagtgaacttatataagttataacaagccttaatcagtactagaccttagcttatactatactacatatacgaattttcgcttttcatattagtatcttactgataagcaataaagtttactacgacatagtaacaataattaactacttgtttcaattcaattagatgatttgggttttgaaatgaatttaacttaaagatgattaccttagatagagtattgttatcttctattaagttaattacttaacttacattaatacatttgttgatattatccaatatccattatcttaaacgaacttacgccatcttgaattagctaatcgacttacattgatacatcgatctaatccaatattaacttgtatcaattcaaattagatgttttgagtttcaatatgcaacttaacttagagacgattaacttaaatgaagcattgttatattgaaccaatctactgagttatactattctacatctgtttgaattagatgctttcggttccgaaatgaattcaacttaaagatgattaacttaagtagagtactgttattatcttgaattaaacttagggtactgttaaactagatgttttgagtttcaatatgaatttaacttagagacgattaacttaaatggagtattgttatattgaaccaatctactgagttccattgttctacatttgttgcaactcaattagatgctttcagttccgaaatgaattcaacttaaaaagatgattaacttaagtagagtactattattatattgaactagtttatcgacttacattgatacactcattcaaataacaacatgtttcaatttaagtaaacgttttgagtttcaattactttatcgacttacgtcgatacattcatctaatctaatatcaattgtaatttcttttagtttggctaagatatgtgagtttcaaattgaatataaattgttagcgattaacttaaatggagtaatgttatcttgatttcacttatcgattcacatagataccttgagctaatccataatcaacttattttacgtcgattatagatatattaatccattgaactaatacactaagagtaagacactaacatagatttgtcttattccaaatcatatatattaagtaataaagtacactttaaccgatgtcactttcattcacgtttttatgaagtacaactatttttttgaggcaattgtggaagtgccgacaacacttatttgttgcattaatttttagccggacccgaaaacacctgaccaaaaccgacatgaagcacaatttttcccgaacaaatacaagagtgtgattaacttctacccgaaccgatgcgattattgtcgaagttatgtggttttttgtcaataactgaaataaaccgaaacgaaactgaaaataaccgcaacaaattttaactgatatggattcaaccgttggttggtttaactgtttggttaaaactgatttaaactgcaactgaactgaactgaaaatggaaagggaaaaaactgaattaaactgaactgaaaagtttgttaactgaactgaactgttaattatttcaactaatacaaactgaactgatatcaactgaactgtttggttataaccataactgaactgttgaactgaattgacacctctaattgcaacggcaacaatagaactgttacagtagcccaatctacaaaactaaattgacaagaactgttgcattagatactcctactacaacggtacgtaataatgtgttgcactagtaaattttagcctttgcctttgattaaaaatgttgtagtgacacatcacaataaaatcaaatctaatgaatatgctataaacaaacaattgttccaaaatcaactattcttacgaaatactcttttcgagggacgacatttttctgaaacaccctaagcgacgttattgactctaatacatagatgaaatatcaataaaaagggatttatatgaggtattctttaatttttttttaagaacatatgaggtaccatagtattttaaaaatcaatccatgtgcccctaacttcgtaatgtatccatcatatatatcccttttttttttaggggtccatcatatatatccttaatgcatgactatcaaacattagatatgtcttaatccccccctaacaatcaaccaaaattccaaaattggttactctatttgttcctattacaccttccaccaatctcaacaaattttggcattctggttaagtactcgaaggactacttaacgaaatatttgatgtttgttagccattaaggtttatgtgttggattcattataaatggtttgaaaagtttattatcatctttattaaaagttcaatagtacctcatgagaactctctataaatcaactatattagttatcaataataagatacattcggattctatttggcaacacatttaagtcacctcaaatgaataggtgttaaaagtttaagtcacctaaaataaatagtcatgattaaagtaagagcttaaatcaaatgtaagctaaaaaattgactgaaattaaattggtatcaagtataacatcttaatttaaggtgttaaaattttcaactaacttatcaattgagcttttttcatcaatttcacctctatttcagctttgtttttagcttatatttccaaaattgccaataggtctcatttatacctaagatattcaacaatcctggctctcttcctgctcgttcattccaaatttcccaaatgtaatcagattaaagcagtagtgatcggatcattgcagacttttcggtagaataactgaataagtgtactgagatagtgtcaaaatactgttatttaagttttctataagtactaaaatatgaattagttcaacatcatcttatattgtcggccaataagattgcccactgtataataatataaaacaaaatcacagaggtgatatcgccttaaaaaaaaaaaatcatagatgtgaattaatgtaaccgagcataaatcaactgtttcctttttttttaaaagagaaatcaactgtttcctagaaacaattgaatattatatggtggtttattgtttcaaatttcagaaaaacaaaaaaaaaaaagcctttgctacttacttacatagttgcatctatataaagggcacctagcccaaccattttacttacctaaactgtctgtgtttgcaccactctcatctaaaaaaacctgtgaaagtgagcgtttcaaaattttcaaaagatggaacgtggagctcccttctctcactatcagctaccaaaatccatctctgaattgaacttggaccagcacagcaacaacccaaccccaatgaccagctcagtcgtagtagccggcgccggtgacaagaacaatggtatcgtggtccagcagcaaccaccatgtgtggctcgtgagcaagaccaatacatgccaatcgcaaacgtcataagaatcatgcgtaaaaccttaccgtctcacgccaaaatctctgacgacgccaaagaaacgattcaagaatgtgtctccgagtacatcagcttcgtgaccggtgaagccaacgagcgttgccaacgtgagcaacgtaagaccataactgctgaagatatcctttgggctatgagcaagcttgggttcgataactacgtggaccccctcaccgtgttcattaaccggtaccgtgagatagagaccgatcgtggttctgcacttagaggtgagccaccgtcgttgagacaaacctatggaggaaatggtattgggtttcacggcccatctcatggcctacctcctccgggtccttatggttatggtatgttggaccaatccatggttatgggaggtggtcggtactaccaaaacgggtcgtcgggtcaagatgaatccagtgttggtggtggctcttcgtcttccattaacggaatgccggcttttgaccattatggtcagtataagtgaagatggaattattcttcatttttatatctgttcaaaacatgtgtttggatagatattttatttttatgtcttatcaataacatttctatataatgttgcttctttaaggaaaagtgttgtatttcaatactttatgagaaactgatttatatatgcaaatgatttaacccaaa

SEQ ID NO: 3 - DNA, 5060 bp, Artificial sequence
  Arabidopsis thaliana and Beta vulgaris
ctcgaggatccttgatttgtttttttgaccaatgagcattaagagtgatcaagcacatggatttcaaaatatcaatggttggatttttatcattctcaaatcaatggttaaagttaaatattctattttctaatttctaaatttataattctatactctagtattgatttgataagcaattagtaatctacttaaattcctaaactattattaataatataaattaataaaaattataaacttagaagtttaattattatttttaatttcaaacaagttgattgatgtaaatatgattatgaatgacgtgaaacagttaaggcaaagttgtagtttagtaactttaaaaattagtagtgttattataacttaatagaaattataaactttcaaaattaaattagtattattgatttttaagaaattattattctaaatttataaattcagaaatatgtgaacatgttacatgcggtgattacggattaatttaatgctaacatgtaagacgtaacacaataacagttaacctactactactacttgtataggtcttacattaatacttaagttattggttataaacttatattaatatacggttatttcttctattaagcgtagatttcttataaccttacttatacaattatttactattactcaactataatttgattgtattcagttaagtgaacttatataagttataacaagccttaatcagtactagaccttagcttatactatactacatatacgaattttcgcttttcatattagtatcttactgataagcaataaagtttactacgacatagtaacaataattaactacttgtttcaattcaattagatgatttgggttttgaaatgaatttaacttaaagatgattaccttagatagagtattgttatcttctattaagttaattacttaacttacattaatacatttgttgatattatccaatatccattatcttaaacgaacttacgccatcttgaattagctaatcgacttacattgatacatcgatctaatccaatattaacttgtatcaattcaaattagatgttttgagtttcaatatgcaacttaacttagagacgattaacttaaatgaagcattgttatattgaaccaatctactgagttatactattctacatctgtttgaattagatgctttcggttccgaaatgaattcaacttaaagatgattaacttaagtagagtactgttattatcttgaattaaacttagggtactgttaaactagatgttttgagtttcaatatgaatttaacttagagacgattaacttaaatggagtattgttatattgaaccaatctactgagttccattgttctacatttgttgcaactcaattagatgctttcagttccgaaatgaattcaacttaaaaagatgattaacttaagtagagtactattattatattgaactagtttatcgacttacattgatacactcattcaaataacaacatgtttcaatttaagtaaacgttttgagtttcaattactttatcgacttacgtcgatacattcatctaatctaatatcaattgtaatttcttttagtttggctaagatatgtgagtttcaaattgaatataaattgttagcgattaacttaaatggagtaatgttatcttgatttcacttatcgattcacatagataccttgagctaatccataatcaacttattttacgtcgattatagatatattaatccattgaactaatacactaagagtaagacactaacatagatttgtcttattccaaatcatatatattaagtaataaagtacactttaaccgatgtcactttcattcacgtttttatgaagtacaactatttttttgaggcaattgtggaagtgccgacaacacttatttgttgcattaatttttagccggacccgaaaacacctgaccaaaaccgacatgaagcacaatttttcccgaacaaatacaagagtgtgattaacttctacccgaaccgatgcgattattgtcgaagttatgtggttttttgtcaataactgaaataaaccgaaacgaaactgaaaataaccgcaacaaattttaactgatatggattcaaccgttggttggtttaactgtttggttaaaactgatttaaactgcaactgaactgaactgaaaatggaaagggaaaaaactgaattaaactgaactgaaaagtttgttaactgaactgaactgttaattatttcaactaatacaaactgaactgatatcaactgaactgtttggttataaccataactgaactgttgaactgaattgacacctctaattgcaacggcaacaatagaactgttacagtagcccaatctacaaaactaaattgacaagaactgttgcattagatactcctactacaacggtacgtaataatgtgttgcactagtaaattttagcctttgcctttgattaaaaatgttgtagtgacacatcacaataaaatcaaatctaatgaatatgctataaacaaacaattgttccaaaatcaactattcttacgaaatactcttttcgagggacgacatttttctgaaacaccctaagcgacgttattgactctaatacatagatgaaatatcaataaaaagggatttatatgaggtattctttaatttttttttaagaacatatgaggtaccatagtattttaaaaatcaatccatgtgcccctaacttcgtaatgtatccatcatatatatcccttttttttttaggggtccatcatatatatccttaatgcatgactatcaaacattagatatgtcttaatccccccctaacaatcaaccaaaattccaaaattggttactctatttgttcctattacaccttccaccaatctcaacaaattttggcattctggttaagtactcgaaggactacttaacgaaatatttgatgtttgttagccattaaggtttatgtgttggattcattataaatggtttgaaaagtttattatcatctttattaaaagttcaatagtacctcatgagaactctctataaatcaactatattagttatcaataataagatacattcggattctatttggcaacacatttaagtcacctcaaatgaataggtgttaaaagtttaagtcacctaaaataaatagtcatgattaaagtaagagcttaaatcaaatgtaagctaaaaaattgactgaaattaaattggtatcaagtataacatcttaatttaaggtgttaaaattttcaactaacttatcaattgagcttttttcatcaatttcacctctatttcagctttgtttttagcttatatttccaaaattgccaataggtctcatttatacctaagatattcaacaatcctggctctcttcctgctcgttcattccaaatttcccaaatgtaatcagattaaagcagtagtgatcggatcattgcagacttttcggtagaataactgaataagtgtactgagatagtgtcaaaatactgttatttaagttttctataagtactaaaatatgaattagttcaacatcatcttatattgtcggccaataagattgcccactgtataataatataaaacaaaatcacagaggtgatatcgccttaaaaaaaaaaaatcatagatgtgaattaatgtaaccgagcataaatcaactgtttcctttttttttaaaagagaaatcaactgtttcctagaaacaattgaatattatatggtggtttattgtttcaaatttcagaaaaacaaaaaaaaaaaagcctttgctacttacttacatagttgcatctatataaagggcacctagcccaaccattttacttacctaaactgtctgtgtttgcaccactctcatctaaaaaaacctgtgaaagtgagcgtttcaaaattttcaaaagatggataacttcttaccctttccctcttctaacgcaaactctgtccaagaactctctatggatcctaacaacaatcgctcgcacttcacaacagtccctacttatgatcatcatcaggctcagcctcatcacttcttgcctccgttttcatacccggtggagcagatggcggcggtgatgaatcctcagccggtttacttatcggagtgttatcctcagatcccggttacgcaaaccggaagtgaattcggttctctggttggtaatccttgtttgtggcaagagagaggtggttttcttgatccgcgtatgacgaagatggcaaggatcaacaggaaaaacgccatgatgagatcaagaaacaactctagccctaattctagtccaagtgagttggttgattcaaagagacagctgatgatgcttaacttgaaaaataacgtgcagatctccgacaagaaagatagctaccaacagtccacatttgataacaagaagcttagggttttgtgtgagaaggaattgaagaacagcgatgttgggtcactcgggaggatagttctaccaaagagagatgcagaagcaaatcttccgaagctatctgataaagaaggaatcgttgtacagatgagagatgttttctctatgcagtcttggtctttcaaatacaagttttggtccaataacaagagcagaatgtatgtcctcgagaacacaggagaatttgtgaagcaaaatggagctgagataggagactttttaacaatatacgaggacgaaagcaagaatctctacttcgccatgaatggaaattcgggaaaacaaaatgaaggaagagaaaatgagtcgagggaaaggaaccactacgaagaggcaatgcttgattacataccaagagacgaagaggaagcttccattgcaatgctcatcggaaatctaaacgatcactatcccatccctaacgatctcatggacctcaccactgaccttcagcaccatcaagccacgtcctcatcaatgccacctgaggatcacgcgtacgtgggttcatccgatgatcaggtgagctttaacgactttgagtggtggtga

SEQ ID NO: 4 - DNA, 5039 bp, Artificial sequence
  Arabidopsis thaliana and Beta vulgaris
ctcgaggatccttgatttgtttttttgaccaatgagcattaagagtgatcaagcacatggatttcaaaatatcaatggttggatttttatcattctcaaatcaatggttaaagttaaatattctattttctaatttctaaatttataattctatactctagtattgatttgataagcaattagtaatctacttaaattcctaaactattattaataatataaattaataaaaattataaacttagaagtttaattattatttttaatttcaaacaagttgattgatgtaaatatgattatgaatgacgtgaaacagttaaggcaaagttgtagtttagtaactttaaaaattagtagtgttattataacttaatagaaattataaactttcaaaattaaattagtattattgatttttaagaaattattattctaaatttataaattcagaaatatgtgaacatgttacatgcggtgattacggattaatttaatgctaacatgtaagacgtaacacaataacagttaacctactactactacttgtataggtcttacattaatacttaagttattggttataaacttatattaatatacggttatttcttctattaagcgtagatttcttataaccttacttatacaattatttactattactcaactataatttgattgtattcagttaagtgaacttatataagttataacaagccttaatcagtactagaccttagcttatactatactacatatacgaattttcgcttttcatattagtatcttactgataagcaataaagtttactacgacatagtaacaataattaactacttgtttcaattcaattagatgatttgggttttgaaatgaatttaacttaaagatgattaccttagatagagtattgttatcttctattaagttaattacttaacttacattaatacatttgttgatattatccaatatccattatcttaaacgaacttacgccatcttgaattagctaatcgacttacattgatacatcgatctaatccaatattaacttgtatcaattcaaattagatgttttgagtttcaatatgcaacttaacttagagacgattaacttaaatgaagcattgttatattgaaccaatctactgagttatactattctacatctgtttgaattagatgctttcggttccgaaatgaattcaacttaaagatgattaacttaagtagagtactgttattatcttgaattaaacttagggtactgttaaactagatgttttgagtttcaatatgaatttaacttagagacgattaacttaaatggagtattgttatattgaaccaatctactgagttccattgttctacatttgttgcaactcaattagatgctttcagttccgaaatgaattcaacttaaaaagatgattaacttaagtagagtactattattatattgaactagtttatcgacttacattgatacactcattcaaataacaacatgtttcaatttaagtaaacgttttgagtttcaattactttatcgacttacgtcgatacattcatctaatctaatatcaattgtaatttcttttagtttggctaagatatgtgagtttcaaattgaatataaattgttagcgattaacttaaatggagtaatgttatcttgatttcacttatcgattcacatagataccttgagctaatccataatcaacttattttacgtcgattatagatatattaatccattgaactaatacactaagagtaagacactaacatagatttgtcttattccaaatcatatatattaagtaataaagtacactttaaccgatgtcactttcattcacgtttttatgaagtacaactatttttttgaggcaattgtggaagtgccgacaacacttatttgttgcattaatttttagccggacccgaaaacacctgaccaaaaccgacatgaagcacaatttttcccgaacaaatacaagagtgtgattaacttctacccgaaccgatgcgattattgtcgaagttatgtggttttttgtcaataactgaaataaaccgaaacgaaactgaaaataaccgcaacaaattttaactgatatggattcaaccgttggttggtttaactgtttggttaaaactgatttaaactgcaactgaactgaactgaaaatggaaagggaaaaaactgaattaaactgaactgaaaagtttgttaactgaactgaactgttaattatttcaactaatacaaactgaactgatatcaactgaactgtttggttataaccataactgaactgttgaactgaattgacacctctaattgcaacggcaacaatagaactgttacagtagcccaatctacaaaactaaattgacaagaactgttgcattagatactcctactacaacggtacgtaataatgtgttgcactagtaaattttagcctttgcctttgattaaaaatgttgtagtgacacatcacaataaaatcaaatctaatgaatatgctataaacaaacaattgttccaaaatcaactattcttacgaaatactcttttcgagggacgacatttttctgaaacaccctaagcgacgttattgactctaatacatagatgaaatatcaataaaaagggatttatatgaggtattctttaatttttttttaagaacatatgaggtaccatagtattttaaaaatcaatccatgtgcccctaacttcgtaatgtatccatcatatatatcccttttttttttaggggtccatcatatatatccttaatgcatgactatcaaacattagatatgtcttaatccccccctaacaatcaaccaaaattccaaaattggttactctatttgttcctattacaccttccaccaatctcaacaaattttggcattctggttaagtactcgaaggactacttaacgaaatatttgatgtttgttagccattaaggtttatgtgttggattcattataaatggtttgaaaagtttattatcatctttattaaaagttcaatagtacctcatgagaactctctataaatcaactatattagttatcaataataagatacattcggattctatttggcaacacatttaagtcacctcaaatgaataggtgttaaaagtttaagtcacctaaaataaatagtcatgattaaagtaagagcttaaatcaaatgtaagctaaaaaattgactgaaattaaattggtatcaagtataacatcttaatttaaggtgttaaaattttcaactaacttatcaattgagcttttttcatcaatttcacctctatttcagctttgtttttagcttatatttccaaaattgccaataggtctcatttatacctaagatattcaacaatcctggctctcttcctgctcgttcattccaaatttcccaaatgtaatcagattaaagcagtagtgatcggatcattgcagacttttcggtagaataactgaataagtgtactgagatagtgtcaaaatactgttatttaagttttctataagtactaaaatatgaattagttcaacatcatcttatattgtcggccaataagattgcccactgtataataatataaaacaaaatcacagaggtgatatcgccttaaaaaaaaaaaatcatagatgtgaattaatgtaaccgagcataaatcaactgtttcctttttttttaaaagagaaatcaactgtttcctagaaacaattgaatattatatggtggtttattgtttcaaatttcagaaaaacaaaaaaaaaaaagcctttgctacttacttacatagttgcatctatataaagggcacctagcccaaccattttacttacctaaactgtctgtgtttgcaccactctcatctaaaaaaacctgtgaaagtgagcgtttcaaaattttcaaaagatggactgggagattcgaggctcatctttgggacaaaagctcttggaattcgattcagaacaagaaaggcaaacaaggtttcgagcatatgacagtgaagaagcagcagcacatacgtacgatctggctgctctcaagtactggggacccgacaccatcttgaattttccggcagagacgtacacaaaggaattggaagaaatgcagagagtgacaaaggaagaatatttggcttctctccgccgccagagcagtggtttctccagaggcgtctctaaatatcgcggcgtcgctaggcatcaccacaacggaagatgggaggctcggatcggaagagtgtttgggaacaagtacttgtacctcggcacctataatacgcaggaggaagctgctgcagcatatgacatggctgcgattgagtatcgaggcgcaaacgcggttactaatttcgacattagtaattacattgaccggttaaagaagaaaggtgttttcccgttccctgtgaaccaagctaaccatcaagagggtattcttgttgaagccaaacaagaagttgaaacgagagaagcgaaggaagagcctagagaagaagtgaaacaacagtacgtggaagaaccaccgcaagaagaagaagagaaggaagaagagaaagcagagcaacaagaagcagagattgtaggatattcagaagaagcagcagtggtcaattgctgcatagactcttcaaccataatggaaatggatcgttgtggggacaacaatgagctggcttggaacttctgtatgatggatacagggttttctccgtttttgactgatcagaatctcgcgaatgagaatcccatagagtatccggagctattcaatgagttagcatttgaggacaacatcgacttcatgttcgatgatgggaagcacgagtgcttgaacttggaaaatctggattgttgcgtggtgggaagagagagcccaccctcttcttcttcaccattgtcttgcttatctactgactctgcttcatcaacaacaacaacaacaacctcggtttcttgtaactatttggtctga

SEQ ID NO: 5 - DNA, 5261 bp, Artificial sequence
  Arabidopsis thaliana and Beta vulgaris
ctcgaggatccttgatttgtttttttgaccaatgagcattaagagtgatcaagcacatggatttcaaaatatcaatggttggatttttatcattctcaaatcaatggttaaagttaaatattctattttctaatttctaaatttataattctatactctagtattgatttgataagcaattagtaatctacttaaattcctaaactattattaataatataaattaataaaaattataaacttagaagtttaattattatttttaatttcaaacaagttgattgatgtaaatatgattatgaatgacgtgaaacagttaaggcaaagttgtagtttagtaactttaaaaattagtagtgttattataacttaatagaaattataaactttcaaaattaaattagtattattgatttttaagaaattattattctaaatttataaattcagaaatatgtgaacatgttacatgcggtgattacggattaatttaatgctaacatgtaagacgtaacacaataacagttaacctactactactacttgtataggtcttacattaatacttaagttattggttataaacttatattaatatacggttatttcttctattaagcgtagatttcttataaccttacttatacaattatttactattactcaactataatttgattgtattcagttaagtgaacttatataagttataacaagccttaatcagtactagaccttagcttatactatactacatatacgaattttcgcttttcatattagtatcttactgataagcaataaagtttactacgacatagtaacaataattaactacttgtttcaattcaattagatgatttgggttttgaaatgaatttaacttaaagatgattaccttagatagagtattgttatcttctattaagttaattacttaacttacattaatacatttgttgatattatccaatatccattatcttaaacgaacttacgccatcttgaattagctaatcgacttacattgatacatcgatctaatccaatattaacttgtatcaattcaaattagatgttttgagtttcaatatgcaacttaacttagagacgattaacttaaatgaagcattgttatattgaaccaatctactgagttatactattctacatctgtttgaattagatgctttcggttccgaaatgaattcaacttaaagatgattaacttaagtagagtactgttattatcttgaattaaacttagggtactgttaaactagatgttttgagtttcaatatgaatttaacttagagacgattaacttaaatggagtattgttatattgaaccaatctactgagttccattgttctacatttgttgcaactcaattagatgctttcagttccgaaatgaattcaacttaaaaagatgattaacttaagtagagtactattattatattgaactagtttatcgacttacattgatacactcattcaaataacaacatgtttcaatttaagtaaacgttttgagtttcaattactttatcgacttacgtcgatacattcatctaatctaatatcaattgtaatttcttttagtttggctaagatatgtgagtttcaaattgaatataaattgttagcgattaacttaaatggagtaatgttatcttgatttcacttatcgattcacatagataccttgagctaatccataatcaacttattttacgtcgattatagatatattaatccattgaactaatacactaagagtaagacactaacatagatttgtcttattccaaatcatatatattaagtaataaagtacactttaaccgatgtcactttcattcacgtttttatgaagtacaactatttttttgaggcaattgtggaagtgccgacaacacttatttgttgcattaatttttagccggacccgaaaacacctgaccaaaaccgacatgaagcacaatttttcccgaacaaatacaagagtgtgattaacttctacccgaaccgatgcgattattgtcgaagttatgtggttttttgtcaataactgaaataaaccgaaacgaaactgaaaataaccgcaacaaattttaactgatatggattcaaccgttggttggtttaactgtttggttaaaactgatttaaactgcaactgaactgaactgaaaatggaaagggaaaaaactgaattaaactgaactgaaaagtttgttaactgaactgaactgttaattatttcaactaatacaaactgaactgatatcaactgaactgtttggttataaccataactgaactgttgaactgaattgacacctctaattgcaacggcaacaatagaactgttacagtagcccaatctacaaaactaaattgacaagaactgttgcattagatactcctactacaacggtacgtaataatgtgttgcactagtaaattttagcctttgcctttgattaaaaatgttgtagtgacacatcacaataaaatcaaatctaatgaatatgctataaacaaacaattgttccaaaatcaactattcttacgaaatactcttttcgagggacgacatttttctgaaacaccctaagcgacgttattgactctaatacatagatgaaatatcaataaaaagggatttatatgaggtattctttaatttttttttaagaacatatgaggtaccatagtattttaaaaatcaatccatgtgcccctaacttcgtaatgtatccatcatatatatcccttttttttttaggggtccatcatatatatccttaatgcatgactatcaaacattagatatgtcttaatccccccctaacaatcaaccaaaattccaaaattggttactctatttgttcctattacaccttccaccaatctcaacaaattttggcattctggttaagtactcgaaggactacttaacgaaatatttgatgtttgttagccattaaggtttatgtgttggattcattataaatggtttgaaaagtttattatcatctttattaaaagttcaatagtacctcatgagaactctctataaatcaactatattagttatcaataataagatacattcggattctatttggcaacacatttaagtcacctcaaatgaataggtgttaaaagtttaagtcacctaaaataaatagtcatgattaaagtaagagcttaaatcaaatgtaagctaaaaaattgactgaaattaaattggtatcaagtataacatcttaatttaaggtgttaaaattttcaactaacttatcaattgagcttttttcatcaatttcacctctatttcagctttgtttttagcttatatttccaaaattgccaataggtctcatttatacctaagatattcaacaatcctggctctcttcctgctcgttcattccaaatttcccaaatgtaatcagattaaagcagtagtgatcggatcattgcagacttttcggtagaataactgaataagtgtactgagatagtgtcaaaatactgttatttaagttttctataagtactaaaatatgaattagttcaacatcatcttatattgtcggccaataagattgcccactgtataataatataaaacaaaatcacagaggtgatatcgccttaaaaaaaaaaaatcatagatgtgaattaatgtaaccgagcataaatcaactgtttcctttttttttaaaagagaaatcaactgtttcctagaaacaattgaatattatatggtggtttattgtttcaaatttcagaaaaacaaaaaaaaaaaagcctttgctacttacttacatagttgcatctatataaagggcacctagcccaaccattttacttacctaaactgtctgtgtttgcaccactctcatctaaaaaaacctgtgaaagtgagcgtttcaaaattttcaaaagatgaagaagcgcttaaccacttccacttgttcttcttctccatcttcctctgtttcttcttctactactacttcctctcctattcagtcggaggctccaaggcctaaacgagccaaaagggctaagaaatcttctccttctggtgataaatctcataacccgacaagccctgcttctacccgacgcagctctatctacagaggagtcactagacatagatggactgggagattcgaggctcatctttgggacaaaagctcttggaattcgattcagaacaagaaaggcaaacaagtttatctgggagcatatgacagtgaagaagcagcagcacatacgtacgatctggctgctctcaagtactggggacccgacaccatcttgaattttccggcagagacgtacacaaaggaattggaagaaatgcagagagtgacaaaggaagaatatttggcttctctccgccgccagagcagtggtttctccagaggcgtctctaaatatcgcggcgtcgctaggcatcaccacaacggaagatgggaggctcggatcggaagagtgtttgggaacaagtacttgtacctcggcacctataatacgcaggaggaagctgctgcagcatatgacatggctgcgattgagtatcgaggcgcaaacgcggttactaatttcgacattagtaattacattgaccggttaaagaagaaaggtgttttcccgttccctgtgaaccaagctaaccatcaagagggtattcttgttgaagccaaacaagaagttgaaacgagagaagcgaaggaagagcctagagaagaagtgaaacaacagtacgtggaagaaccaccgcaagaagaagaagagaaggaagaagagaaagcagagcaacaagaagcagagattgtaggatattcagaagaagcagcagtggtcaattgctgcatagactcttcaaccataatggaaatggatcgttgtggggacaacaatgagctggcttggaacttctgtatgatggatacagggttttctccgtttttgactgatcagaatctcgcgaatgagaatcccatagagtatccggagctattcaatgagttagcatttgaggacaacatcgacttcatgttcgatgatgggaagcacgagtgcttgaacttggaaaatctggattgttgcgtggtgggaagagagagcccaccctcttcttcttcaccattgtcttgcttatctactgactctgcttcatcaacaacaacaacaacaacctcggtttcttgtaactatttggtctga

SEQ ID NO: 6 - DNA, 1135 bp, Manihot esculenta
ggtgatgagaagagagactatttcgttgacaaaaagaaacgataaggatatttaatagatatgaaaaaagataaagacattttaatatatttatgaaaatatagggagggacgatttcgttgacggaaagaaacgatgaggagagactatttcattgacggaaagaaatgatgagaagggactatttcgttgacggaaagaaacgatgaggatggactatttcgttaacggaaagaaacgataaggacgttttaatagatatgagaaaagataaggactatttcattgacggaaagaaacataaggacgttttactagatatgaaaaagataaaaacgttttaatagatttttgaaaatatatggactaacttgttaataatagtaatatccaggactaaataaggggttttaattgagggtaaaattggatttttaaacattttctctagcctcctttatttaattttaatgaaaaagaggacggaaggactatttcgttgacggaaaaaaaatataaggacgttttaataggtttctaaaaatatagggactgacttgttaataatgacaataatcagggactaaattgtaaatctcccaatatatatttactatttagaaagcattaatatattatatattttcacagttgataataattgatgacgaagaaatctcatggatctagctacaagatcaacttgtttaacattagtatcaaccattttgccatttctctttgatttcagtgagatgaggggtcaaatcccaagattcctctttcttaagtgctcccacctcgttctctttcatacatgaacttctggcccttcaattctctatataagccactcttattcatcctctctctgcaccatctctctctttctgtctctccttcctgtttgcttctcagctttattttttagtttctatttccttggctgactatggctactgctgaggtaacccatcaatcatttcttgttaagctttgattcaggttcttgattttaattattgatctcattagtttcagcagctttacatgataatgaaagaattttatcttaaagatcttttgatgaattttgattttaggtagtaacagcacagactgcacttcctgagga

SEQ ID NO: 7 - DNA, 2044 bp, Artificial sequence
  Arabidopsis thaliana and Manihot esculenta
ggtgatgagaagagagactatttcgttgacaaaaagaaacgataaggatatttaatagatatgaaaaaagataaagacattttaatatatttatgaaaatatagggagggacgatttcgttgacggaaagaaacgatgaggagagactatttcattgacggaaagaaatgatgagaagggactatttcgttgacggaaagaaacgatgaggatggactatttcgttaacggaaagaaacgataaggacgttttaatagatatgagaaaagataaggactatttcattgacggaaagaaacataaggacgttttactagatatgaaaaagataaaaacgttttaatagatttttgaaaatatatggactaacttgttaataatagtaatatccaggactaaataaggggttttaattgagggtaaaattggatttttaaacattttctctagcctcctttatttaattttaatgaaaaagaggacggaaggactatttcgttgacggaaaaaaaatataaggacgttttaataggtttctaaaaatatagggactgacttgttaataatgacaataatcagggactaaattgtaaatctcccaatatatatttactatttagaaagcattaatatattatatattttcacagttgataataattgatgacgaagaaatctcatggatctagctacaagatcaacttgtttaacattagtatcaaccattttgccatttctctttgatttcagtgagatgaggggtcaaatcccaagattcctctttcttaagtgctcccacctcgttctctttcatacatgaacttctggcccttcaattctctatataagccactcttattcatcctctctctgcaccatctctctctttctgtctctccttcctgtttgcttctcagctttattttttagtttctatttccttggctgactatggataacttcttaccctttccctcttctaacgcaaactctgtccaagaactctctatggatcctaacaacaatcgctcgcacttcacaacagtccctacttatgatcatcatcaggctcagcctcatcacttcttgcctccgttttcatacccggtggagcagatggcggcggtgatgaatcctcagccggtttacttatcggagtgttatcctcagatcccggttacgcaaaccggaagtgaattcggttctctggttggtaatccttgtttgtggcaagagagaggtggttttcttgatccgcgtatgacgaagatggcaaggatcaacaggaaaaacgccatgatgagatcaagaaacaactctagccctaattctagtccaagtgagttggttgattcaaagagacagctgatgatgcttaacttgaaaaataacgtgcagatctccgacaagaaagatagctaccaacagtccacatttgataacaagaagcttagggttttgtgtgagaaggaattgaagaacagcgatgttgggtcactcgggaggatagttctaccaaagagagatgcagaagcaaatcttccgaagctatctgataaagaaggaatcgttgtacagatgagagatgttttctctatgcagtcttggtctttcaaatacaagttttggtccaataacaagagcagaatgtatgtcctcgagaacacaggagaatttgtgaagcaaaatggagctgagataggagactttttaacaatatacgaggacgaaagcaagaatctctacttcgccatgaatggaaattcgggaaaacaaaatgaaggaagagaaaatgagtcgagggaaaggaaccactacgaagaggcaatgcttgattacataccaagagacgaagaggaagcttccattgcaatgctcatcggaaatctaaacgatcactatcccatccctaacgatctcatggacctcaccactgaccttcagcaccatcaagccacgtcctcatcaatgccacctgaggatcacgcgtacgtgggttcatccgatgatcaggtgagctttaacgactttgagtggtggtga

(19) United States
(12) Patent Application Publication
(10) Pub. No.: US 2010/0257639 A1
(43) Pub. Date: Oct. 7, 2010

(54) METHODS AND COMPOSITIONS FOR ALTERING SUGAR BEET OR ROOT CROP STORAGE TISSUE

(76) Inventors: Robert Edward Bruccoleri, Glastonbury, CT (US); Peter Raynolds Berger, II, San Francisco, CA (US)

Correspondence Address: JONDLE & ASSOCIATES P.C., 858 HAPPY CANYON ROAD SUITE 230, CASTLE ROCK, CO, 80108, US

(21) Appl. No.: 12/712,926

(22) Filed: Feb. 25, 2010

Related U.S. Application Data
(60) Provisional application No. 61/155,773, filed on Feb. 26, 2009.
(60) Provisional application No. 61/227,856, filed on Jul. 23, 2009.

(51) Int. Cl. A01H 5/00; A01G 1/00

(52) U.S. Cl. 800/298; 47/58.1 FV

(57) ABSTRACT

Methods and compositions for altering sugar beet or root crop storage tissue are disclosed. The invention relates to the alteration of certain pathways in sugar beet or root crops to promote storage of certain fats and oils instead of sugar.

### CROSS-REFERENCE TO RELATED APPLICATIONS

This application is a non-provisional application of U.S. Provisional Patent Application No. 61/155,773, filed on Feb. 26, 2009, and U.S. Provisional Patent Application No. 61/227,856, filed on Jul. 23, 2009, which are herein incorporated by reference in their entirety.

### BACKGROUND

The present invention relates to methods and compositions for altering sugar beet or root crop storage tissue. More specifically, the present invention relates to the modification of biochemical pathways expressed in sugar beet or root crop root storage parenchyma to produce fats or oils instead of storing sucrose. All publications, GenBank accessions, and other references cited in this application are herein incorporated by reference.

Global warming, fossil fuel depletion, and the growth of worldwide energy consumption are major issues facing the 21st century. Governments and companies worldwide have been searching for ways to deal with these issues, in particular searching for ways to increase production of alternative fuels. Using corn to produce ethanol is one way of producing an alternative fuel. Another way is using enzymes to increase the breakdown of cellulose for conversion to ethanol.

The use of modern day crops is not sufficient to produce enough fuel to meet the increasing worldwide demand due to a lack of optimization for fuel production. For example, ethanol from corn uses a small fraction of the total corn plant mass for fuel, and significant energy losses are incurred by the fermentation and distillation processes.

In contrast, plant fats (also referred to as oils or lipids) are chemically much more similar to crude oil. Most plant fats are esters of glycerol and long chain fatty acids. Fatty acids are hydrocarbons (approximately 16 carbons) with a carboxylic acid group at one end. The long chain hydrocarbons are similar to the aliphatic fraction crude oil with the difference being the carboxylic acid group. Plant fats are highly compatible with existing petroleum infrastructure. They can be transported in existing pipelines with no modifications, and reactors are available at commercial refinery scales to reduce the carboxylic acid group at low cost. Thus, the cost to transport and refine plant fatty acids will be small.

The use of plant fats is already commercialized by the biodiesel industry. Here, agricultural plant oils such as cooking oils (soybean, canola, sunflower, etc.) are chemically treated to convert them into fuel. The plant oils are fatty acids which have been biochemically coupled to glycerin. The biodiesel processor performs a transesterification reaction which removes the glycerin and replaces it with an ethyl ester. These esters can be used instead of diesel fuel.

The foregoing examples of the related art and limitations related therewith are intended to be illustrative and not exclusive. Other limitations of the related art will become apparent to those of skill in the art upon a reading of the specification.

### SUMMARY OF THE INVENTION

The following embodiments and aspects thereof are described in conjunction with systems, tools, and methods, which are meant to be exemplary, not limiting in scope. In various embodiments, one or more of the above-described problems have been reduced or eliminated, while other embodiments are directed to other improvements.

The present invention provides methods and compositions for altering sugar beet or root crop storage tissue.

In one aspect of the present invention, a method for altering storage tissue in a root crop plant is provided which comprises growing a transgenic root crop plant having a DNA construct stably integrated into its genome under conditions suitable for the expression of the DNA construct in the storage tissue. The DNA construct expresses a transcription factor in said storage tissue when compared to said storage tissue in a non-transgenic plant, where the transcription factor alters the expression of other gene products within the storage tissue.

In another aspect of the present invention, a transgenic sugar beet plant is provided comprising a DNA construct stably integrated into its genome. The DNA construct is capable of expression in a sugar beet root storage parenchyma, where the DNA construct is capable of expressing a transcriptional factor when compared to a non-transgenic sugar beet plant, wherein the transcriptional control protein alters production of fatty acids in the parenchyma.

It is another aspect of the present invention where a DNA construct is provided that comprises a promoter operable in the storage tissue of the root crop plant operably linked to a nucleic acid capable of expressing said transcription factor.

It is another aspect of the present invention wherein a DNA construct is provided which comprises a nucleic acid having the sequence of SEQ ID NO:2 or a nucleic acid having at least 70% identity to the sequence of SEQ ID NO:2.

It is another aspect of the present invention where a DNA construct is provided that comprises a nucleic acid having the sequence of SEQ ID NO:3 or a nucleic acid having at least 70% identity to the sequence of SEQ ID NO:3.

It is another aspect of the present invention where a DNA construct is provided that comprises a nucleic acid having the sequence of SEQ ID NO:4 or a nucleic acid having at least 70% identity to the sequence of SEQ ID NO:4.

It is another aspect of the present invention where a DNA construct is provided that comprises a nucleic acid having the sequence of SEQ ID NO:5 or a nucleic acid having at least 70% identity to the sequence of SEQ ID NO:5.

It is another aspect of the present invention where a DNA construct is provided in a transgenic sugar beet comprising a promoter operable in the parenchyma operably linked to a nucleic acid capable of expressing a transcription factor when compared to a non-transgenic sugar beet plant.

It is another aspect of the present invention where a DNA construct is provided in a transgenic sugar beet comprising a nucleic acid having the sequence of SEQ ID NO:2 or a nucleic acid having at least 70% identity to the sequence of SEQ ID NO:2.

It is another aspect of the present invention where a DNA construct is provided in a transgenic sugar beet comprising a nucleic acid having the sequence of SEQ ID NO:3 or a nucleic acid having at least 70% identity to the sequence of SEQ ID NO:3.

It is another aspect of the present invention where a DNA construct is provided in a transgenic sugar beet comprising a nucleic acid having the sequence of SEQ ID NO:4 or a nucleic acid having at least 70% identity to the sequence of SEQ ID NO:4.

It is another aspect of the present invention where a DNA construct is provided in a transgenic sugar beet comprising a nucleic acid having the sequence of SEQ ID NO:5 or a nucleic acid having at least 70% identity to the sequence of SEQ ID NO:5.

It is yet another aspect of the present invention to provide a method for altering root crop plant storage tissue which comprises growing a transgenic root crop plant having a DNA construct stably integrated into its genome under conditions suitable for the expression of the DNA construct in the storage tissue. The DNA construct expresses a transcription factor when compared to a non-transgenic root crop plant, and the transcription factor increases the production of fatty acids in the storage tissue.

It is another aspect of the present invention to provide a DNA construct in a transgenic root crop plant comprising a promoter operable in the storage tissue of the root crop plant operably linked to a nucleic acid capable of expressing the transcription factor.

It is another aspect of the present invention to provide a DNA construct in a transgenic root crop plant comprising a nucleic acid having the sequence of SEQ ID NO:2 or a nucleic acid having at least 70% identity to the sequence of SEQ ID NO:2.

It is another aspect of the present invention where a DNA construct in a transgenic root crop plant is provided comprising a nucleic acid having the sequence of SEQ ID NO:3 or a nucleic acid having at least 70% identity to the sequence of SEQ ID NO:3.

It is another aspect of the present invention where a DNA construct in a transgenic root crop plant is provided comprising a nucleic acid having the sequence of SEQ ID NO:4 or a nucleic acid having at least 70% identity to the sequence of SEQ ID NO:4.

It is another aspect of the present invention where a DNA construct in a transgenic root crop plant is provided comprising a nucleic acid having the sequence of SEQ ID NO:5 or a nucleic acid having at least 70% identity to the sequence of SEQ ID NO:5.

It is another aspect of the present invention where a transgenic root crop plant is provided comprising a DNA construct stably integrated into its genome under conditions suitable for the expression of the DNA construct in storage tissue of the root crop plant. The DNA construct expresses a transcription factor when compared to a non-transgenic root crop plant, and the transcription factor increases the production of fatty acids in the storage tissue.

It is another aspect of the present invention where a DNA construct in a transgenic root crop is provided comprising a promoter operable in the storage tissue operably linked to a nucleic acid capable of altering the expression level of the transcription factor.

It is another aspect of the present invention where a DNA construct in a transgenic root crop is provided comprising a nucleic acid having the sequence of SEQ ID NO:2 or a nucleic acid having at least 70% identity to the sequence of SEQ ID NO:2.

It is another aspect of the present invention where a DNA construct in a transgenic root crop is provided comprising a nucleic acid having the sequence of SEQ ID NO:3 or a nucleic acid having at least 70% identity to the sequence of SEQ ID NO:3.

It is another aspect of the present invention where a DNA construct in a transgenic root crop is provided comprising a nucleic acid having the sequence of SEQ ID NO:4 or a nucleic acid having at least 70% identity to the sequence of SEQ ID NO:4.

It is another aspect of the present invention where a DNA construct in a transgenic root crop is provided comprising a nucleic acid having the sequence of SEQ ID NO:5 or a nucleic acid having at least 70% identity to the sequence of SEQ ID NO:5.

In one aspect of the present invention, the LEC1 gene is used in a construct to alter at least one biochemical pathway expressed in sugar beet or root crop root storage parenchyma. LEC1 genes are well known to the skilled artisan and include, but are not limited to, Arabidopsis thaliana LEC1 (GenBank Accession No. NM 102046), Zea mays (GenBank Accession No. AF410176.1 or NM_{—}001112048.1), Oryza sativa (GenBank Accession No. AY264284), and the like.

In another aspect of the present invention, the LEC2 gene is used in a construct to alter at least one biochemical pathway expressed in sugar beet or root crop root storage parenchyma. LEC2 genes are well known to the skilled artisan and include, but are not limited to, Arabidopsis thaliana (GenBank Accession No. NM_{—}102595.2 or AY568668.1), and the like.

In another aspect of the present invention, the Wrinkled (WRI1) gene is used in a construct to alter at least one biochemical pathway expressed in sugar beet or root crop root storage tissue of the parenchyma. WRI1 genes are well known to the skilled artisan and include, but are not limited to, Arabidopsis thaliana (GenBank Accession No. NM 202701.2, NM_{—}115292.4, or NM_{—}001035780.2), Brassica napus (GenBank Accession No. DQ370141.1 or DQ402050.1), Zea mays (GenBank Accession No. EU960249.1), and the like.

In another aspect of the present invention, sugar beet or root crop plants are transformed such that at least one biochemical pathway expressed in sugar beet or root crop root storage tissue of the parenchyma is modified to produce fats instead of storing sugar.

In yet another aspect of the present invention, said transformed sugar beet or root crop plants are used to produce progeny sugar beet or root crop plants having the at least one altered biochemical pathway.

In addition to the exemplary aspects and embodiments described above, further aspects and embodiments will become apparent by study of the following descriptions.

### BRIEF DESCRIPTION OF THE FIGURES

FIG. 1a is a photomicrograph of sugar beet taproot.

FIG. 1b is a photomicrograph of sugar beet taproot displaying expression pattern of AX449164.

### SUMMARY OF THE SEQUENCE LISTING

SEQ ID NO:1 sets forth the sequence of the major latex-like protein promoter, GenBank Accession No. AX449164.

SEQ ID NO:2 sets forth the sequence of a modified fragment of the AX449164 promoter of SEQ ID NO:1 and the LEC1 gene fused to create the construct of SEQ ID NO:2.

SEQ ID NO:3 sets forth the sequence of a modified fragment of the AX449164 promoter of SEQ ID NO:1 and the LEC2 gene fused to create the construct of SEQ ID NO:3.

SEQ ID NO:4 sets forth the sequence of a modified fragment of the AX449164 promoter of SEQ ID NO:1 and one splice variant of the Wrinkled gene fused to create the construct of SEQ ID NO:4.

SEQ ID NO:5 sets forth the sequence of a modified fragment of the AX449164 promoter of SEQ ID NO:1 and a second splice variant of the Wrinkled gene fused to create the construct of SEQ ID NO:5.

SEQ ID NO:6 sets forth the sequence of the glutamic acid rich protein promoter, GenBank Accession No. FJ688171.

SEQ ID NO:7 sets forth the sequence of a modified fragment of the FJ688171 promoter of SEQ ID NO:6 and the LEC2 gene fused to create the construct of SEQ ID NO:7.

### DEFINITIONS

In order to provide a clear and consistent understanding of the specification and claims, including the scope to be given terms that may be used in the specification and claims, the following definitions are provided:

Allele. An allele is any of one or more alternative forms of a gene, which relate to one trait or characteristic. In a diploid cell or organism, the two alleles of a given gene occupy corresponding loci on a pair of homologous chromosomes.

Alter. The utilization of up-regulation, down-regulation, or gene silencing.

Biochemical pathway. As used herein, a “biological pathway” means the organizational units of metabolism the pathways that energy and materials follow in the cell.

Biodiesel. As used herein, “biodiesel” means plant oils which have been transesterified with a short chain alcohol and which can be used directly in diesel fuel engines.

Biological replicates. Multiple samples in a biological experiment that have all been treated in the same way. For example, consider an experiment to evaluate the hypothesis that administration of insulin causes reduction in blood sugar. Each experimental subject after the first in such an experiment would be a biological replicate.

Cambium. A layer of undifferentiated cells in a plant that divide and differentiate into other plant tissues.

Cell. As used herein, “cell” includes a plant cell, whether isolated, in tissue culture, or incorporated in a plant or plant part.

Construct. As used herein, a “construct” is an artificially constructed segment of DNA that may be transplanted into a target plant tissue or plant cell.

Enzyme. As used herein, an “enzyme” is one or more proteins that catalyze a chemical reaction in a plant tissue or plant cell.

Essentially all the physiological and morphological characteristics. A plant having “essentially all the physiological and morphological characteristics” means a plant having all of its physiological and morphological characteristics, except for the characteristics derived from the converted gene.

Expression. As used herein, the term “expression” includes the process by which information from a gene is used in the synthesis of a functional gene product, such as the expression of fatty acids in the storage tissue of root crops. These products are often proteins, but in non-protein coding genes such as rRNA genes or tRNA genes, the product is a functional RNA. The process of gene expression is used by all known life, i.e., eukaryotes (including multicellular organisms), prokaryotes (bacteria and archaea), and viruses, to generate the macromolecular machinery for life. Several steps in the gene expression process may be modulated, including the transcription, up-regulation, RNA splicing, translation, and post-translational modification of a protein.

Fatty acid. As used herein, “fatty acid” means oils, fats, or lipids that are composed of chains of carbon and hydrogen atoms, with a carboxylic acid group at one end or such chains with the carboxylic acid reacted with a hydroxyl group of glyercol to make an ester linkage.

Feedstock. As used herein, “feedstock” means a substitute for petroleum for a refinery that is economically comparable to crude oil.

Gene. As used herein, “gene” refers to a segment of nucleic acid. A gene can be introduced into a genome of a species, whether from a different species or from the same species, using transformation or various breeding methods.

Gene expression variability. The variability across biological replicates of specific messenger RNA's as measured using transcriptional profiling. Gene expression can be highly variable among different samples of the same tissues.

Gene product. As used herein, “gene product” means biochemical material resulting from the expression of a gene, including, but not limited to, RNA or proteins.

Gene silencing. The interruption or suppression of the expression of a gene at the level of transcription or translation.

Genotype. Refers to the genetic constitution of a cell or organism.

Homologous recombination. A genetic event where two double-stranded DNA molecules of similar or identical sequence are enzymatically broken and rejoined so that one of the two strands in one of the DNA molecules is exchanged with the corresponding strand in the other molecule.

Linkage. “Linkage” refers to a phenomenon wherein alleles on the same chromosome tend to segregate together more often than expected by chance if their transmission was independent.

Linkage disequilibrium. “Linkage disequilibrium” refers to a phenomenon wherein alleles tend to remain together in linkage groups when segregating from parents to offspring, with a greater frequency than expected from their individual frequencies.

Locus. A locus confers one or more traits such as, for example, male sterility, herbicide tolerance, insect resistance, disease resistance, waxy starch, modified fatty acid metabolism, modified phytic acid metabolism, modified carbohydrate metabolism, and modified protein metabolism. The trait may be, for example, conferred by a naturally occurring gene introduced into the genome of the variety by backcrossing, a natural or induced mutation, or a transgene introduced through genetic transformation techniques. A locus may comprise one or more alleles integrated at a single chromosomal location.

Multiplex identifier kits. A set of reagents used in sequencing that allows the user to mix multiple samples together in a single sequencing run and still be able to identify each sample when the DNA sequences are determined.

Percent identity. Percent identity as used herein with respect to a comparison of two plant varieties refers to the comparison of the homozygous alleles of two plant varieties. Percent identity is determined by comparing a statistically significant number of the homozygous alleles of two developed varieties. For example, a percent identity of 90% between plant variety 1 and plant variety 2 means that the two varieties have the same allele at 90% of their loci. Percent identity as used herein with respect to two nucleic acids refers to the comparison of the entire sequence for each of the two nucleic acids and is determined by GAP alignment using default parameters (GCG, GAP version 10, Accelrys, San Diego, Calif.). GAP uses the algorithm of Needleman and Wunsch, J Mol Biol, 48:443-453 (1970), to find the alignment of two complete sequences that maximizes the number of matches and minimizes the number of sequence gaps. Sequences which have 100% identity are identical. The present invention encompasses nucleic acids that have about 70%, 75%, 80%, 85%, 90%, 91%, 92%, 93%, 94%, 95%, 96%, 97%, 98%, 99%, or 100% sequence identity to the specified nucleic acid.

Percent similarity. Percent similarity as used herein refers to the comparison of the homozygous alleles of one plant variety with those of another plant, and if the homozygous allele of the first plant matches at least one of the alleles from the other plant then they are scored as similar. Percent similarity is determined by comparing a statistically significant number of loci and recording the number of loci with similar alleles as a percentage. A percent similarity of 90% between the first plant and a second plant means that the first matches at least one of the alleles of the second plant at 90% of the loci.

Plant. As used herein, the term “plant” includes reference to an immature or mature whole plant, including a plant from which seed, grain, anthers, or pistils have been removed. A seed or embryo that will produce the plant is also considered to be the plant.

Plant parts. As used herein, the term “plant parts” (or a sugar beet or root crop plant, or a part thereof) includes protoplasts, leaves, stems, roots, root tips, anthers, seed, embryo, pollen, ovules, cotyledon, hypocotyl, flower, shoot, tissue, petiole, cells, meristematic cells, and the like.

Promoter. A used herein, a “promoter” is a DNA region, which includes sequences sufficient to cause transcription of an associated (downstream) sequence. The promoter may be regulated, i.e., not constitutively acting to cause transcription of the associated sequence. If inducible, there are sequences present therein which mediate regulation of expression so that the associated sequence is transcribed only when an inducer molecule is present. The promoter may be any DNA sequence which shows transcriptional activity in the chosen plant cells, plant parts, or plants. The promoter may be inducible or constitutive. It may be naturally-occurring, may be composed of portions of various naturally-occurring promoters, or may be partially or totally synthetic. Guidance for the design of promoters is provided by studies of promoter structure, such as that of Harley and Reynolds, Nucleic Acids Res., 15, 2343-61 (1987). Also, the location of the promoter relative to the transcription start may be optimized. Many suitable promoters for use in plants are well known in the art, as are nucleotide sequences, which enhance expression of an associated expressible sequence.

Quantitative trait loci (QTL). Quantitative trait loci (QTL) refer to genetic loci that control to some degree numerically representable traits that are usually continuously distributed.

Regeneration. Regeneration refers to the development of a plant from tissue culture.

Root crop. As used herein, the term “root crop” means a plant that is cultivated primarily for its roots such as, but not limited to, cassava, taro, beets, turnips, carrots, potatoes, yams, rutabagas, radishes, jicama, or parsnips.

Root vasculature. Tissues in the plant root that are responsible for carrying nutrients to and from the root. These tissues are generally tubular.

Single gene converted (conversion). Single gene converted (conversion) plants refers to plants which are developed by a plant breeding technique called backcrossing wherein essentially all of the desired morphological and physiological characteristics of a variety are recovered in addition to the single gene transferred into the variety via the backcrossing technique or via genetic engineering. It also refers to plants, which are developed through either naturally occurring mutation or induced mutation.

Storage tissues. In the sugar beet or root crop root, there are specific tissues for the storage of sugar. These tissues are physically and visually distinct from the root vasculature.

Sugar beet. As used herein, “sugar beet” means a plant, or a part thereof, of Beta vulgaris.

Taproot. A very large root used for storing plant nutrients. Sugar beets, carrots, red beets, radishes, and parsnips are all examples of plants having taproots.

Targeting constructs. Engineered DNA molecules that encode specific genes and flanking sequences that enable the constructs to integrate into the host genome at specific (targeted) locations. Targeting constructs depend upon homologous recombination to find their targets.

Transcriptome. As used herein, “transcriptome” means all the messenger RNA sequences produced in a cell or tissue at a specific developmental stage.

Transcription factor. As used herein, “transcription factor” is a protein that binds to a specific DNA sequence and thereby controls the transfer (or transcription) of genetic information from DNA to mRNA. Transcription factors perform this function alone or with other proteins in a complex, by promoting (as an activator), or blocking (as a repressor) the recruitment of RNA polymerase (the enzyme that performs the transcription of genetic information from DNA to RNA) to specific genes.

Transgenic. As used herein, “transgenic” is used to indicate a plant, or photosynthetic organism including algae, which has been genetically modified to contain the DNA constructs of the present invention.

Up-regulation. As used herein, “up-regulation” means a process by which occurs within a cell triggered by a signal (originating internal or external to the cell) which results in increased expression of one or more genes and as a result the protein(s) or gene products encoded by those genes as compared to a cell without up-regulation.

### DETAILED DESCRIPTION OF THE INVENTION

The present invention provides methods and compositions to engineer sugar beet or root crop plants to efficiently make high quality oil feedstocks for the petroleum industry from carbon dioxide and water using solar energy at a cost superior to crude oil extracted from the earth. The present invention also provides examples of regulatory pathway genetics needed to engineer sugar beet or root crop plants to produce large quantities of fats in the sugar beet or root crop taproot. The present invention identifies sugar beet or root crop genomic regions which contain transcripts that are expressed only in the sugar storage regions of the taproot, and which can be modified to express additional genes in just those sugar storage regions.

As a class of agricultural products, root crops have nearly an order of magnitude higher yield of harvested weight per acre than any other crop—approximately 20-30 tons per acre. The cost of production of root crops is low, about $0.021 per pound for sugar beets, which includes the farmer's profit. For any part of the United States, there are root crops which can be grown.

The sucrose storage tissue in the taproot is an anatomically distinct region from the root vasculature and cambium layer of the root (Artschwager, E., “Anatomy of the Vegetative Organs of the Sugar Beet,” J. Ag. Res., 33(2):143-176 (1926)). In principle, plants can be optimized for fuel production by genetically modifying them to produce oil directly in a form that can be delivered to a refinery. For example, the up-regulation of the LEC2 gene or diacylglycerolacyltransferase (DGAT) has been demonstrated to increase the production of fatty acids in the leaves of tobacco (Andrianov, W., et al., Plant Biotechnology Journal, Vol. 8, pp. 1-11 (2009)). For another example, Arabidopsis thaliana has been transformed to make omega-3 and omega-6 fatty acids (Qi, B., et al., Nature Biotechnology, 22(6):739-745 (2004)).

Existing oil refineries have the ability to economically remove carboxylic acid groups from hydrocarbons. Also, relatively simple chemical refinement (transesterification) can efficiently transform fatty acids into biodiesel.

The efficiencies of using sugar beet or root crops for oil production are very favorable compared to producing ethanol from corn. In 2007, corn yields averaged 151 bushels per acre and the average yield of ethanol per bushel was 2.7 gallons, thus 408 gallons of ethanol/acre. In contrast, an acre of sugar beets can produce 7000 pounds of sugar. If this quantity of product was fat and converted to biodiesel (using a conversion factor of 7 pounds/gallon of diesel fuel), it would yield approximately 1000 gallons of fuel, a 2.5 fold improvement over ethanol. This factor ignores the higher energy density of diesel versus ethanol. Economically, the comparison is also favorable. The cost per gallon for the agricultural product input for sugar beets is approximately $1.05/gallon. The cost of corn for one gallon of ethanol is approximately $1.48, and the cost of fermentation and distillation would be higher than the extraction from fat laden sugar beets.

Most importantly, the present invention has some similarities to the overarching concept of self-replicating machines (see U.S. Pat. No. 6,510,359, “Method and system for self-replicating manufacturing stations”), except that plants are the self-replicating chemical factories. The inputs to these factories are solar energy, carbon dioxide, water, and fertilizer, and the output is liquid fuel in the form of long chain fatty acids. Unexpectedly, the important contrast to all other biofuel inventions is that the present invention uses the plant for as much chemistry as possible, so no additional chemical process infrastructure is needed. Only new facilities needed to extract oils from the sugar beet or root crop roots will be needed. Thus, the cost per gallon of fuel will be minimized relative to fermentation or algal biofuel methods.

Because the present invention uses carbon dioxide from the atmosphere as the carbon source for the production of oil in the taproot of the plant, and because the amount of oil that can be produced from an acre of land has far more energy than the amount needed to make the fertilizer to grow an acre of sugar beets, this invention has a negative carbon footprint. Thus, the exclusive use of this invention for all liquid fuel needs will reduce the growth in greenhouse gases from liquid fuels.

The present invention results in the modification of the biochemical pathways expressed in the sugar beet or root crop root storage tissue of the parenchyma to produce fats instead of storing sucrose. The sugar beet or root crop root storage tissues are transformed to up-regulate sucrose degradation enzymes and fatty acid biosynthesis pathways by duplicating those genes into constructs that have storage tissue specific promoters. Since the root vasculature and the cambium layer are both distinct from the storage tissues, such modifications are not lethal.

In the native sugar beet plant (prior to any selective breeding to increase sucrose yield), the taproot is used as a food source for the plant for the second year growth which produces flowers. One might imagine that the modification of the taproot to make fats would prevent flowering. However, mutant sugar beets have been identified that do not require large taproot formation for flowering (see Panella, et al., Crop Science, 35:1721 (1995)). Thus modified sugar beet plants will reproduce normally even with a taproot storing fat. In addition, the genetic ancestors of modern sugar beets had much lower concentrations of sugar, and they were able to make seeds. Alternatively, inducible promoters can be used so a fraction of the plants can reproduce.

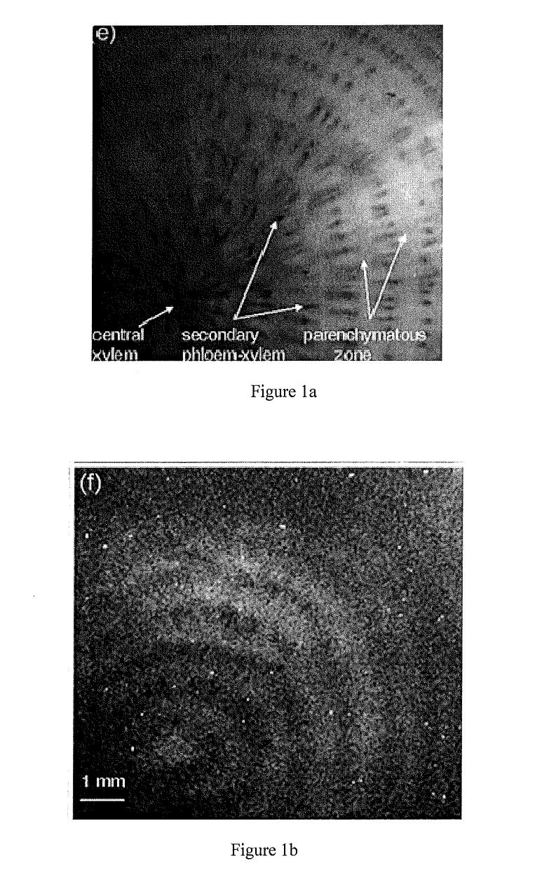

The present invention includes the identification of genes specifically expressed in the storage tissues in the sugar beet or root crop taproot and in no other location of the plant. In order to identify these genes, plant tissues outside of the storage tissues are sampled for comparison. Four tissue samples are taken from four different regions of the sugar beet or root crop plant. The first sample is taken from the tips of the leaves of the sugar beet. The second sample is taken from the white part of the sugar beet leaf stems starting one inch above the root crown. The third sample is taken from the small roots emanating from the taproot. The fourth sample is taken from the flowering buds of the sugar beet. For the sugar beet taproot, samples from both immature roots (roots less than or equal to one centimeter in diameter) and mature roots are taken. From the immature roots and mature roots, samples are taken from the outer one millimeter of the taproot, storage parenchyma (parenchymatous zone), and vascular tissue (central xylem) for a total of ten tissue samples (tissue types are shown in FIG. 1a).

Three biological replicates are use to estimate gene expression variability. A total of thirty samples for sequencing with two samples per plate are expressed using multiplex identifier kits. The total number of sequenced base pairs is 2.4 gigabase pairs based upon the equation (30 samples/2 samples per plate)×(400,000 reads per plate)×(400 base pairs per read). For a sample of the preparation protocol see Eveland, et al., “Transcript Profiling by 3′-Untranslated Region Sequencing Resolves Expression of Gene Families,” Plant Physiology, 146:32-44 (2008). As shown in the Eveland protocol, specific tissues from the plant are dissected and placed in RNAlater®, a reagent sold by Ambion®. The tissues are then processed using the RNeasy® kit (Qiagen®) to extract the RNA. The RNA is then reverse transcribed into DNA for use in sequencing. The sequencing of the transcriptome is performed using the published procedure of 454 Life Sciences Corporation.

The sugar beet genome is estimated at 720 Mbps. The sugar beet genome is sequenced using Roche 454 sequencing technology. In order to carry out shotgun sequencing, large-size genomic DNA samples are randomly fragmented into small 300- to 800-base-pair fragments via physical shearing. Addition of adapters to the generated fragments created a library of DNA fragments which are immobilized on DNA capture beads and individually sequenced on a PicoTiterPlate device. The generated sequences are then assembled into a number of unordered and unoriented contigs, a set of overlapping DNA segments derived from a single genetic source using the GS De Novo Assembler Software and a consensus sequence is generated.

After assembly of de novo shotgun sequencing reads into contigs, the generated contigs are ordered and oriented using paired-end reads. These paired-end reads have two 100-mer DNA segments on each side (paired ends) that were originally located at either the 3 kb, 8 kb, or kb apart in the sequence of interest. The GS De Novo Assembler Software enables subsequent mapping of the 100-mer fragments to the generated contigs and thus ordering and orienting of the contigs into scaffolds. This combined information provides a high-quality draft sequence of the genome.

An alternate genome sequencing approach that is available is the use of the Illumina Solexa technology. In order to shotgun sequence the sugar beet genome, large-size genomic DNA samples are randomly fragmented into small 300- to 800-base-pair fragments via physical shearing. The fragments are attached to the glass plates of the Illumina sequencing system and are amplified in place on the plate. These fragments are then used as templates for step-wise single nucleotide additions with fluorescent terminators that can be read using microscopy. Subsequent additions read each of the successive base pair fragments. There are multiple software systems that can assemble data from one Illumina sequencing system into much larger contiguous sequences from which gene coding sequences can be identified.

Once the sugar beet genome is sequenced, the sequenced transcripts are mapped onto the annotated genome using sequence similarity algorithms. Storage parenchyma specific transcripts that map uniquely to the genome are then identified. The mapping of transcripts is done using the BLAST sequence similarity algorithm as implemented by the NCBI Toolkit. The goal of using the BLAST sequence similarity algorithm is to find a segmentation of each transcript where each segment matches the genome exactly in one location and where the segments appear in the same order in both the transcript and the genome. The genomic sequence between each transcript segment represents an intron, and the matching segments are exons. Techniques for handling multiple matches and mismatches are well-known in the art.

Generally the DNA that is introduced into a plant is part of a construct. The DNA may be a gene of interest, e.g., a coding sequence for a protein, or it may be a sequence that is capable of regulating expression of a gene, such as an antisense sequence, a sense suppression sequence, or a miRNA sequence. The construct typically includes regulatory regions operatively linked to the 5′ side of the DNA of interest and/or to the 3′ side of the DNA of interest. A cassette containing all of these elements is also referred to herein as an expression cassette. The expression cassettes may additionally contain 5′ leader sequences in the expression cassette construct. The regulatory regions (i.e., promoters, transcriptional regulatory regions, and translational termination regions) and/or the polynucleotide encoding a signal anchor may be native/analogous to the host cell or to each other. Alternatively, the regulatory regions and/or the polynucleotide encoding a signal anchor may be heterologous to the host cell or to each other. See, U.S. Pat. No. 7,205,453 and U.S. Patent Application Publication Nos. 2006/0218670 and 2006/0248616. The expression cassette may additionally contain selectable marker genes. See, U.S. Pat. No. 7,205,453 and U.S. Patent Application Publication Nos. 2006/0218670 and 2006/0248616.

Generally, the expression cassette will comprise a selectable marker gene for the selection of transformed cells. Selectable marker genes are utilized for the selection of transformed cells or tissues. Usually, the plant selectable marker gene will encode antibiotic resistance, with suitable genes including at least one set of genes coding for resistance to the antibiotic spectinomycin, the streptomycin phosphotransferase (spt) gene coding for streptomycin resistance, the neomycin phosphotransferase (nptII) gene encoding kanamycin or geneticin resistance, the hygromycin phosphotransferase (hpt or aphiv) gene encoding resistance to hygromycin, acetolactate synthase (als) genes. Alternatively, the plant selectable marker gene will encode herbicide resistance, such as resistance to the sulfonylurea*type herbicides, glufosinate, glyphosate, ammonium, bromoxynil, imidazolinones, and 2,4-dichlorophenoxyacetate (2,4-D), including genes coding for resistance to herbicides which act to inhibit the action of glutamine synthase, such as phosphinothricin or basta (e.g., the bar gene). See generally, WO 02/36782, U.S. Pat. No. 7,205,453 and U.S. Patent Application Publication Nos. 2006/0248616 and 2007/0143880, and those references cited therein. This list of selectable marker genes is not meant to be limiting. Any selectable marker gene can be used.

A number of promoters can be used in the practice of the invention. The promoters can be selected based on the desired outcome. That is, the nucleic acids can be combined with constitutive, tissue-preferred, or other promoters for expression in the host cell of interest. The promoter of the present invention may be any DNA sequence, which shows transcriptional activity in the chosen plant cells, plant parts, or plants. The promoter may be inducible or constitutive. It may be naturally-occurring, may be composed of portions of various naturally-occurring promoters, or may be partially or totally synthetic. Guidance for the design of promoters is provided by studies of promoter structure, such as that of Harley and Reynolds, Nucleic Acids Res. 15:2343-61 (1987). Also, the location of the promoter relative to the transcription start may be optimized. Many suitable promoters for use in plants are well known in the art, as are nucleotide sequences, which enhance expression of an associated expressible sequence.

For instance, suitable constitutive promoters for use in plants include promoters from plant viruses, such as the peanut chlorotic streak caulimovirus (PC1SV) promoter (U.S. Pat. No. 5,850,019), the 35S promoter from cauliflower mosaic virus (CaMV) (Odell, et al., Nature, 313:810-812 (1985)), promoters of Chlorella virus methyltransferase genes (U.S. Pat. No. 5,563,328), and the full-length transcript promoter from figwort mosaic virus (FMV) (U.S. Pat. No. 5,378,619); the promoters from such genes as rice actin (McElroy, et al., Plant Cell, 2:163-171 (1990)), ubiquitin (Christensen, et al., Plant Mol. Biol., 12:619-632 (1989) and Christensen, et al., Plant Mol. Biol., 18:675-689 (1992)), pEMU (Last, et al., Theor. Appl. Genet. 81:581-588 (1991)), MAS (Velten, et al., EMBO J., 3:2723-2730 (1984)), maize H3 histone (Lepetit, et al., Mol. Gen. Genet., 231:276-285 (1992) and Atanassova, et al., Plant Journal, 2(3):291-300 (1992)), Brassica napus ALS3 (WO 97/41228); and promoters of various Agrobacterium genes (see U.S. Pat. Nos. 4,771,002, 5,102,796, 5,182,200, and 5,428,147). Finally, promoters composed of portions of other promoters and partially or totally synthetic promoters can be used. See, e.g., Ni, et al., Plant J., 7:661-676 (1995) and WO 95/14098 describing such promoters for use in plants. Other constitutive promoters include, for example, those disclosed in U.S. Pat. Nos. 5,608,149, 5,608,144, 5,604,121, 5,569,597, 5,466,785, 5,399,680, 5,268,463, and 5,608,142.

Other promoters include inducible promoters, particularly from a pathogen-inducible promoter. Such promoters include those from pathogenesis-related proteins (PR proteins), which are induced following infection by a pathogen (e.g., PR proteins, SAR proteins, beta-1,3-glucanase, chitinase, etc.). Other promoters include those that are expressed locally at or near the site of pathogen infection. In further embodiments, the promoter may be a wound-inducible promoter. In other embodiments, chemical-regulated promoters can be used to modulate the expression of a gene in a plant through the application of an exogenous chemical regulator. The promoter may be a chemical-inducible promoter, where application of the chemical induces gene expression, or a chemical-repressible promoter, where application of the chemical represses gene expression. In addition, tissue-preferred promoters can be utilized to target enhanced expression of a polynucleotide of interest within a particular plant tissue. Each of these promoters are described in U.S. Pat. Nos. 6,506,962, 6,575,814, 6,972,349, and 7,301,069 and in U.S. Patent Application Publication Nos. 2007/0061917 and 2007/0143880.

The promoter may include, or be modified to include, one or more enhancer elements. Preferably, the promoter will include a plurality of enhancer elements. Promoters containing enhancer elements provide for higher levels of transcription as compared to promoters that do not include them. Suitable enhancer elements for use in plants include the PC1SV enhancer element (U.S. Pat. No. 5,850,019), the CaMV 35S enhancer element (U.S. Pat. Nos. 5,106,739 and 5,164,316), and the FMV enhancer element (Maiti, et al., Transgenic Res., 6:143-156 (1997)). See also WO 96/23898 and Enhancers And Eukaryotic Expression (Cold Spring Harbor Press, Cold Spring Harbor, N.Y. (1983)).

An example of a storage tissue specific promoter that may be used in the present invention is the major latex-like protein promoter, a known storage tissue specific promoter (Oltmanns, H., et al., “Taproot promoters cause tissue specific gene expression within the storage root of sugar beet,” Planta, 224: 485-495 (2006), GenBank Accession No. AX449164), also set forth in SEQ ID NO:1. The major latex like protein in sugar beet or root crops has sequence similarity to the major latex proteins from the opium poppy, but the function of the protein in sugar beet or root crops is not completely understood. This promoter drives expression of proteins within the storage parenchyma, albeit weakly.

In some embodiments, the DNA of interest is involved in a biochemical pathway which is modified in the sugar beet or root crop to prevent sugar storage and promote fat storage in the taproot. Suitable pathways are the sucrose degradation pathway, the fatty acid biosynthesis pathway, the fatty acid control pathway regulated by the LEC1 transcription factor, the fatty acid control pathway regulated by the LEC2 transcription factor, and the fatty acid control pathway regulated by the WRI1 transcription factor. Thus, examples of suitable genes include a LEC1 gene, a LEC2 gene, or a WRI1 gene. These genes have been shown to induce fatty acid biosynthesis throughout Arabidopsis tissues when these genes were overexpressed in the plant. The up-regulation of the LEC2 gene or diacylgycerolacyltransferase (DGAT) has been demonstrated to increase the production of fatty acids in the leaves of tobacco (Andrianov, W., et al., “Tobacco as a production platform for biofuel: overexpression of Arabidopsis DGAT and LEC2 genes increases accumulation and shifts the composition of lipids in green biomass,” Plant Biotechnology Journal, Vol. 8, pp. 1-11 (2009). Andrianov and coworkers demonstrated that the Arabidopsis LEC2 gene functions within tobacco cells. Thus, the Arabidopsis LEC2 gene, LEC1 gene, and Wrinkled gene can function in the sugar beet or root crop root.

The up-regulation of the LEC2 gene has been shown (Che, N., et al., “Efficient LEC2 activation of OLEOSIN expression requires two neighboring RY elements on its promoter,” Science in China Series C: Life Sciences, pp. 854-863 (2009) to also up-regulate the Oleosin gene, which is used by plants to stabilize fats and oils into a globule. Thus, the up-regulation of the LEC2 will not only increase the production of fats and oils, but will also induce the production of a structural protein, Oleosin, that will stabilize the additional fats and oils produced.

In one aspect, the present invention combines the major latex-like promoter with the LEC1 gene and/or the major latex-like promoter with the LEC2 gene and/or the major latex-like promoter with splice variants of the Wrinkled gene to cause an up-regulation of all fatty acid biosynthetic pathways in the storage cells of the sugar beet or root crop, and nowhere else in the sugar beet or root crop plant, thereby consuming the sucrose delivered by the leaves and converting it into fatty acids while not affecting the growth of the plant as a whole.

Where appropriate, the DNA of interest may be optimized for increased expression in the transformed plant. That is, the coding sequences can be synthesized using plant-preferred codons for improved expression. Methods are available in the art for synthesizing plant-preferred genes. See, for example, U.S. Pat. Nos. 5,380,831, 5,436,391, and 7,205,453 and U.S. Patent Application Publication Nos. 2006/0218670 and 2006/0248616.

Sugar beet or other root crops can be transformed with the expression constructs using techniques well known to the skilled artisan. For example, sugar beet can be transformed using Agrobacterium-mediated transformation, such as disclosed by D'Halluin, et al., “Transformation of Sugar beet (Beta vulgaris L.) and Evaluation of Herbicide Resistance in Transgenic Plants,” Nature Biotechnology, 10:309-314 (1992), Lindsey ang Gallois, “Transformation of Sugar beet (Beta vulgaris) by Agrobacterium tumefaciens,” J Exp Botany, 41:529-536 (1990), or Kishchenko, et al., “Production of transgenetic sugar beet (Beta vulgaris L.) plants resistant to phosphinothricin,” Cell Biology International, 29:15-19 (2005). Alternatively, sugar beet can be transformed using a polyethylene glycol-mediated transformation, such as disclosed by Hall, et al., “A high efficiency technique for the generation of transgenic sugar beets from stomatal guard cells,” Nature Biotechnology, 14:1133-1138 (1996). In addition, sugar beet can be transformed using particle bombardment, such as disclosed by U.S. Pat. No. 6,114,603 or Ivic and Smigocki, “Transformation of Sugar Beet Cell Suspension Cultures,” In Vitro Cellular & Developmental Biology, 39:573-577 (2005). Additional methods for sugar beet or root crop transformation are described by Gurel, et al., Critical Reviews in Plant Sciences, 27:108-140 (2008).

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis, et al., Molecular Cloning, Cold Spring Harbor Laboratory Press, Cold Spring Harbor, N.Y. (1982); Sambrook, et al., Molecular Cloning, 2nd Ed., Cold Spring Harbor Laboratory Press, Cold Spring Harbor, N.Y. (1989); Sambrook and Russell, Molecular Cloning, 3rd Ed., Cold Spring Harbor Laboratory Press, Cold Spring Harbor, N.Y. (2001); Ausubel, et al., Current Protocols in Molecular Biology, John Wiley & Sons (including periodic updates) (1992); Glover, DNA Cloning, IRL Press, Oxford (1985); Russell, Molecular biology of plants: a laboratory course manual, Cold Spring Harbor Laboratory Press, Cold Spring Harbor, N.Y. (1984); Anand, Techniques for the Analysis of Complex Genomes, Academic Press, NY (1992); Guthrie and Fink, Guide to Yeast Genetics and Molecular Biology, Academic Press, NY (1991); Harlow and Lane, Antibodies, Cold Spring Harbor Laboratory Press, Cold Spring Harbor, N.Y. (1988); Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, A. R. Liss, Inc. (1987); Immobilized Cells And Enzymes, IRL Press (1986); B. Perbal, A Practical Guide To Molecular Cloning (1984); the treatise, Methods In Enzymology, Academic Press, Inc., NY); Methods In Enzymology, Vols. 154 and 155, Wu, et al., eds.; Immunochemical Methods In Cell And Molecular Biology, Mayer and Walker, eds., Academic Press, London (1987); Handbook Of Experimental Immunology, Volumes I-IV, D. M. Weir and C. C. Blackwell, eds. (1986); Riott, Essential Immunology, 6th Edition, Blackwell Scientific Publications, Oxford (1988); Fire, et al., RNA Interference Technology: From Basic Science to Drug Development, Cambridge University Press, Cambridge (2005); Schepers, RNA Interference in Practice, Wiley-VCH (2005); Engelke, RNA Interference (RNAi): The Nuts & Bolts of siRNA Technology, DNA Press (2003); Gott, RNA Interference, Editing, and Modification: Methods and Protocols (Methods in Molecular Biology), Human Press, Totowa, N.J. (2004); and Sohail, Gene Silencing by RNA Interference: Technology and Application, CRC (2004).

### EXAMPLES

The following examples are provided to further illustrate the present invention and are not intended to limit the invention beyond the limitations set forth in the appended claims.

### Example 1

### A LEC1 Targeted Construct

The present invention provides an example of four synthesized constructs. The first gene construct example is set forth in SEQ ID NO:2. This gene construct is made by fusing the 5′ end of the sugar beet major latex-like protein promoter, e.g., nucleotides 1-3933 of SEQ ID NO:1, to nucleotides 24-58 of the sugar beet major latex-like protein (GenBank Accession No. AJ309170.1), followed by the complete coding region for Leafy Cotyledon) (LEC1) (Mu, J., et al., “LEAFY COTYLEDON1 Is a Key Regulator of Fatty Acid Biosynthesis in Arabidopsis,” Plant Physiology, Vol. 148, pp. 1042-1054 (2008)). The sequence for LEC1 gene in SEQ ID NO:2 is taken from NCBI Refseq NM_{—}102046.4.

### Example 2

### A LEC2 Targeted Construct

An example of the second gene construct is set forth in SEQ ID NO:3. This construct is a fusion of the 5′ end of the sugar beet major latex-like protein promoter, e.g., nucleotides 1-3933 of SEQ ID NO:1, to nucleotides 24-58 of the sugar beet major latex-like protein (GenBank Accession No. AJ309170.1), followed by the complete coding region for the Leafy Cotyledon2 (LEC2) (Mendoza, M., et al., “LEAFY COTYLEDON 2 activation is sufficient to trigger the accumulation of oil and seed specific mRNAs in Arabidopsis leaves,” FEBS Letters, 579, pp. 4666-4670 (2005)). The sequence for LEC2 in SEQ ID NO:3 is taken from NCBI Refseq NM_{—}102595.2.

### Example 3

### WRI1 Targeted Constructs

Third and fourth examples of gene constructs are set forth in SEQ ID NO:4 and SEQ ID NO:5. These constructs are fusions of two splice variants of the WRI1 gene, also known as the Wrinkled gene. These constructs are a fusion of the 5′ end of the sugar beet major latex-like protein promoter, e.g., nucleotides 1-3933 of SEQ ID NO:1, to nucleotides 24-58 of the sugar beet major latex-like protein (GenBank Accession No. AJ309170.1), followed by the complete coding region for two splice variants of the Wrinkled gene (Cernac, et al., “WRINKLED1 encodes an AP2/EREB domain protein involved in the control of storage compound biosynthesis in Arabidopsis,” The Plant Journal, 40, 575-585 (2004)). The sequence for Wrinkled1 in SEQ ID NO:4 is taken from NCBI Refseq NM_{—}202701.2. The sequence for Wrinkled1 in SEQ ID NO:5 is taken from NCBI Refseq NM_{—}001035780.2.

### Example 4

### A Cassava LEC2 Targeted Construct

The present invention also provides an example of the expression of fatty acids in the root storage tissue of other root crops such as a cassava plant. As an example, a synthesized construct for use in a cassava root plant is provided. The cassava gene construct is set forth in SEQ ID NO:7. This construct is a fusion of the 5′ end of the cassava protein promoter, e.g., nucleotides 1-1135 of SEQ ID NO:6 (GenBank Accession No. FJ688171), followed by the complete coding region for the Leafy Cotyledon2 (LEC2) (Mendoza, M., et al., “LEAFY COTYLEDON 2 activation is sufficient to trigger the accumulation of oil and seed specific mRNAs in Arabidopsis leaves,” FEBS Letters, 579, pp. 4666-4670 (2005)). The sequence for LEC2 in SEQ ID NO:7 is taken from NCBI Refseq NM_{—}102595.2.

### Example 5

### Examples of Additional Targeted Constructs

Further targeted constructs are made by selecting other genes to alter the metabolic pathways of the sugar beet or other root crop. Examples of such targeted constructs include the following:
1. DNA sequence AX449164 fused to the sugar beet vacuolar invertase gene.
2. DNA sequence AX449164 fused to the sugar beet acid invertase gene.
3. DNA sequence AX449164 fused to the sugar beet diacylglyercol transferase gene.
4. DNA sequence AX449164 fused to the sugar beet acetyl CoA carboxylase gene.
5. DNA sequence AX449164 fused to the sugar beet acetyl-CoA ACP (Acyl Carrier Protein) transacyclase gene.
6. DNA sequence AX449164 fused to the sugar beet malonyl CoA:ACP transacyclase gene.
7. DNA sequence AX449164 fused to the sugar beet short-chain condensing enzyme (KAS III).
8. DNA sequence AX449164 fused to the sugar beet β-ketoacyl-ACP synthetase I gene.
9. DNA sequence AX449164 fused to the sugar beet β-ketoacyl-ACP synthetase II gene.
10. DNA sequence AX449164 fused to the sugar beet β-ketoacyl-ACP reductase gene.
11. DNA sequence AX449164 fused to the sugar beet β-ketoacyl-ACP dehydrase gene.
12. DNA sequence AX449164 fused to the sugar beet enoyl-ACP reductase gene
13. DNA sequence AX449164 fused to the sugar beet oleosin gene.

The first two constructs shown above cause the transformed sugar beet root to hydrolyze sucrose into glucose and fructose, which enter the glycolytic pathway. The next ten constructs cause the transformed sugar beet root to synthesize the fatty acid synthesis multienzyme complex resulting in production of fatty acids from the glycolytic products of glucose and fructose. The final construct produces the oleosin protein which stabilizes the fats and oils that are produced when the fatty acids are coupled to glycerin using the enzymes that normally present within cells.

These thirteen constructs are grouped into sequences of 100 kilobases or less. The constructs in each group are linked together and inserted into different Agrobacterium tumefaciens plasmids with different selection markers. All of these Agrobacterium tumefaciens strains are used to transform sugar beets with a final plant being made that has all 13 constructs incorporated into its genome.

### Example 6

### Transformation of Sugar Beet

Transgenic sugar beet plants that contain the gene of interest, such as those described above, are obtained using conventional technology. For example, transgenic sugar beets are obtained using the method described by Norouzi, et al., “Using a competent tissue for efficient transformation of sugarbeet (Beta vulgaris 1.),” In Vitro Cell. Dev. Biol. Plant, Vol. 41, pp. 11-16 (2005), which describes in detail a cell culture procedure for transforming sugar beets that had a high rate of success.

Alternatively, transgenic sugar beets are obtained using the method described in U.S. Pat. No. 5,969,215, entitled a “Method of plant tissue culture and regeneration” incorporated by reference herein for this purpose. With this method, leaf tissue is macerated and possibly sonicated to remove most cell types except guard cells. The cell walls of these guard cells are digested with cellulases. Uptake of DNA constructs can be mediated using polyethylene glycol, and then selection conditions can be applied to select for transformed guard cells. Callus can be grown from the protoplasts and regenerants will appear after eight weeks of culture.

In addition, transgenic sugar beets are obtained using particle bombardment as described in U.S. Pat. No. 5,969,215, incorporated by reference herein for this purpose. Gold particle bombardment is used to transform the guard cells while in the intact leaf. Leaf tissue is macerated and possibly sonicated to remove most cell types except guard cells. The cell walls of these guard cells are digested with cellulases. Selection conditions are be applied to select for transformed guard cells. Callus can be grown from the protoplasts and regenerants will appear after eight weeks of culture.

Further, transgenic sugar beets are obtained using Agrobacterium-mediated transformation and somatic embryogenesis as described in U.S. Pat. No. 6,555,375, “Methods for Somatic Embryo Formation and Plant Regeneration of Beta Vulgaris” incorporated by reference herein for this purpose. Using the fourth method, small pieces of sugar beet or root crop plants are excised and the excised pieces are treated with specific media that induce formation of callus cells, which are undifferentiated tissue masses. In addition to the callus formation media, the explants are treated with engineered Agrobacterium tumifaciens. After a specified period of time, the cells undergo selection treatments that kill any cells that have not been transformed. The calli are treated with another medium that induces proliferation, which results in embryo-like masses. These are then treated with a regeneration medium, which induces new plants to form.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions, and sub-combinations as are within their true spirit and scope.

The use of the terms “a,” “an,” and “the,” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

We claim:

1. A method for altering storage tissue in a root crop plant which comprises growing a transgenic root crop plant having a DNA construct stably integrated into its genome under conditions suitable for the expression of the DNA construct in the storage tissue, wherein the DNA construct expresses a transcription factor in said storage tissue when compared to said storage tissue in a non-transgenic plant, wherein the transcription factor alters the expression of other gene products within the storage tissue.

2. The method of claim 1, wherein the root crop plant is sugar beet.

3. The method of claim 1, wherein the DNA construct comprises a promoter operable in the storage tissue of the root crop plant operably linked to a nucleic acid capable of expressing said transcription factor.

4. The method of claim 3, wherein said DNA construct comprises a nucleic acid having the sequence of SEQ ID NO:2 or a nucleic acid having at least 70% identity to the sequence of SEQ ID NO:2.

5. The method of claim 3, wherein said DNA construct comprises a nucleic acid having the sequence of SEQ ID NO:3 or a nucleic acid having at least 70% identity to the sequence of SEQ ID NO:3.

6. The method of claim 3, wherein said DNA construct comprises a nucleic acid having the sequence of SEQ ID NO:4 or a nucleic acid having at least 70% identity to the sequence of SEQ ID NO:4.

7. The method of claim 3, wherein said DNA construct comprises a nucleic acid having the sequence of SEQ ID NO:5 or a nucleic acid having at least 70% identity to the sequence of SEQ ID NO:5.

8. A transgenic sugar beet plant comprising a DNA construct stably integrated into its genome, wherein the DNA construct is capable of expression in a sugar beet root storage parenchyma, wherein the DNA construct is capable of expressing a transcription factor when compared to a non-transgenic sugar beet plant, wherein the transcription factor alters production of fatty acids in the parenchyma.

9. The transgenic sugar beet of claim 8, wherein the DNA construct comprises a promoter operable in the parenchyma operably linked to a nucleic acid capable of expressing a transcription factor when compared to a non-transgenic sugar beet plant.

10. The transgenic sugar beet plant of claim 9, wherein said DNA construct comprises a nucleic acid having the sequence of SEQ ID NO:2 or a nucleic acid having at least 70% identity to the sequence of SEQ ID NO:2.

11. The transgenic sugar beet plant of claim 9, wherein said DNA construct comprises a nucleic acid having the sequence of SEQ ID NO:3 or a nucleic acid having at least 70% identity to the sequence of SEQ ID NO:3.

12. The transgenic sugar beet plant of claim 9, wherein said DNA construct comprises a nucleic acid having the sequence of SEQ ID NO:4 or a nucleic acid having at least 70% identity to the sequence of SEQ ID NO:4.

13. The transgenic sugar beet plant of claim 9, wherein said DNA construct comprises a nucleic acid having the sequence of SEQ ID NO:5 or a nucleic acid having at least 70% identity to the sequence of SEQ ID NO:5.

14. A method for altering root crop plant storage tissue which comprises growing a transgenic root crop plant having a DNA construct stably integrated into its genome under conditions suitable for the expression of the DNA construct in the storage tissue, wherein the DNA construct expresses a transcription factor when compared to a non-transgenic root crop plant, wherein the transcription factor increases the production of fatty acids in the storage tissue.

15. The method of claim 14, wherein the DNA construct comprises a promoter operable in the storage tissue of the root crop plant operably linked to a nucleic acid capable of expressing the transcription factor.

16. The method of claim 15, wherein the DNA construct comprises a nucleic acid having the sequence of SEQ ID NO:2 or a nucleic acid having at least 70% identity to the sequence of SEQ ID NO:2.

17. The method of claim 15, wherein the DNA construct comprises a nucleic acid having the sequence of SEQ ID NO:3 or a nucleic acid having at least 70% identity to the sequence of SEQ ID NO:3.

18. The method of claim 15, wherein the DNA construct comprises a nucleic acid having the sequence of SEQ ID NO:4 or a nucleic acid having at least 70% identity to the sequence of SEQ ID NO:4.

19. The method of claim 15, wherein the DNA construct comprises a nucleic acid having the sequence of SEQ ID NO:5 or a nucleic acid having at least 70% identity to the sequence of SEQ ID NO:5.

20. A transgenic root crop plant comprising a DNA construct stably integrated into its genome under conditions suitable for the expression of the DNA construct in storage tissue of the root crop plant, wherein the DNA construct expresses a transcription factor when compared to a non-transgenic root crop plant, wherein the transcription factor increases the production of fatty acids in the storage tissue.

21. The transgenic root crop plant of claim 20, wherein the DNA construct comprises a promoter operable in the storage tissue operably linked to a nucleic acid capable of altering the expression level of the transcription factor.

22. The transgenic root crop plant of claim 21, wherein the DNA construct comprises a nucleic acid having the sequence of SEQ ID NO:2 or a nucleic acid having at least 70% identity to the sequence of SEQ ID NO:2.

23. The transgenic root crop plant of claim 21, wherein the DNA construct comprises a nucleic acid having the sequence of SEQ ID NO:3 or a nucleic acid having at least 70% identity to the sequence of SEQ ID NO:3.

24. The transgenic root crop plant of claim 21, wherein the DNA construct comprises a nucleic acid having the sequence of SEQ ID NO:4 or a nucleic acid having at least 70% identity to the sequence of SEQ ID NO:4.

25. The transgenic root crop plant of claim 21, wherein the DNA construct comprises a nucleic acid having the sequence of SEQ ID NO:5 or a nucleic acid having at least 70% identity to the sequence of SEQ ID NO:5.
